(19) United States
(12) Patent Application Publication
(10) Pub. No.: US 2017/0327789 A1
(43) Pub. Date: Nov. 16, 2017

(54) Modulation of Dopamine Receptor to Promote Neural Cell Differentiation

(71) Applicant: The Hospital for Sick Children, Toronto (CA)

(72) Inventors: Peter Dirks, Toronto (CA); Sonam Dolma, Toronto (CA)

(21) Appl. No.: 15/526,105

(22) PCT Filed: Nov. 13, 2015
(86) PCT No.: PCT/CA2015/051186

§ 371 Date: May 11, 2017

Related U.S. Application Data
(60) Provisional application No. 62/079,725, filed on Nov. 14, 2014.

(51) Int. Cl. C12N 5/0797; A61K 31/352; C07D 311/76; C07D 413/04; C12N 5/0793; A61K 31/454

(52) U.S. Cl. CPC C12N 5/0623; C12N 5/0619; A61K 31/454; C07D 311/76; C07D 413/04; A61K 31/352; C12N 2501/115; C12N 2501/815; C12N 2533/32; C12N 2533/52

(57) ABSTRACT

A method of promoting neural stem cell differentiation is provided comprising exposing neural stem cells to a dopamine D₄ receptor antagonist.

### FIELD OF THE INVENTION

The present invention generally relates to cell differentiation, and more particularly relates to modulation of dopamine receptors to promote neural cell differentiation and to treat neurodegenerative disease.

### BACKGROUND OF THE INVENTION

Neurotransmitters are endogenous chemical messengers that mediate the synaptic function of differentiated neural cells in the mature CNS. Recent studies suggest an important role of neuro chemicals, for example gamma-aminobutyric acid (GABA) and glutamate, in regulating NSC fate both in early development and in adult neurogenesis. GABA regulates adult mouse hippocampal NSCs by maintaining their quiescence through the GABA_{A} receptor, yet can also promote embryonic NSC proliferation, suggesting context specific functions. These effects may reflect influences of local or more distant neuronal activity on the NSC niche. Consistent with this idea, dopamine afferents project to neurogenic zones and depletion of dopamine decreases the proliferation of progenitor cells in the adult subventricular zone (SVZ) through D2-like receptors. Dopamine is also present in early neuronal development in the lateral ganglionic eminence (LGE) and modulates LGE progenitor cell proliferation. Serotonin signaling similarly contributes to the SVZ NSC niche.

It would be desirable to develop a method of differentiating cells which may be useful to treat pathogenic conditions.

### SUMMARY OF THE INVENTION

It has now been determined that dopamine receptor D4 (DRD4) antagonists have utility with respect to differentiation of neural stein cells.

Thus, in one aspect of the present invention, a method of promoting mammalian neural stem cell differentiation is provided comprising exposing neural stem cells to a DRD4 antagonist.

In another aspect of the invention, a method of promoting differentiation of neural stem cells into glutamatergic cells is provided comprising exposing neural stem cells to a DRD4 antagonist.

In a further aspect of the invention, a method of promoting differentiation of neural stem cells into GABAergic cells is provided comprising exposing neural stem cells to a DRD4 antagonist.

In a further aspect of the invention, a method of treating a neurodegenerative disease in a mammal is provided, comprising administering to the mammal a DRD4 antagonist.

These and other aspects of the invention are described herein by reference to the following figures.

### BRIEF DESCRIPTION OF THE FIGURES

FIG. 1 is a schematic illustrating the method used to identify compounds that cause differentiation of neural stem cells into specific neuronal lineages;

FIG. 2 illustrates that dopamine D4 receptor antagonists promote glutamatergic neuron differentiation in neural stem cells;

FIG. 3 graphically illustrates that treatment of neural stem cells with dopamine D4 receptor antagonist, L-741,742, resulted in cells exhibiting increased Vglut1 (A) and Neurog2 (B) expression;

FIG. 4 graphically illustrates that treatment of neural stem cells with dopamine D4 receptor antagonist, L-741,742, resulted in cells exhibiting increased expression of the telencephalic marker, Foxg1 (A) and the cortical layer V marker, Ctip2 (B);

FIG. 5 graphically illustrates that treatment of neural stem cells with dopamine D4 receptor antagonist, L-741,742, resulted in cells exhibiting increased GABA expression;

FIG. 6 is a schematic illustrating a single cell Fluidigm array analysis method used to further analyze differentiated cells;

FIG. 7 graphically illustrates the results of the Fluidigm analysis of L-741,742 differentiated cells confirming the presence of additional glutamatergic markers including CamkII, Vglut1, Vglut2 and Vglut3; and

FIG. 8 illustrates the nucleic acid-encoding sequence (A) and amino acid sequence (B) of DRD4.

### DETAILED DESCRIPTION OF THE INVENTION

In one aspect of the invention, a method of promoting mammalian neural stern cell differentiation is provided comprising exposing mammalian neural stern cells to a DRD4 antagonist.

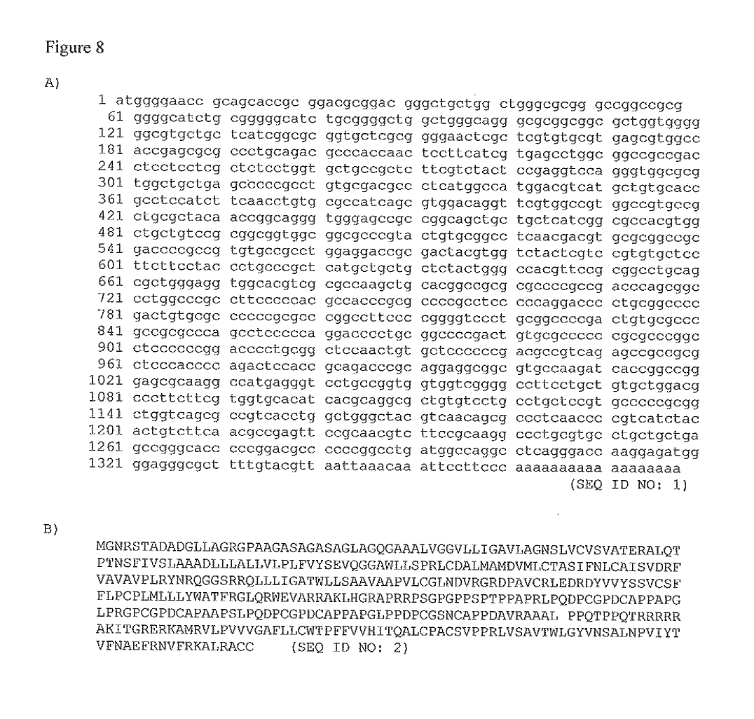

The term “DRD4”, or dopamine receptor D₄, is a G protein-coupled receptor. As with other dopamine receptor subtypes, the D₄ receptor is activated by the neurotransmitter dopamine. The D₄ receptor is D₂-like in that the activated D₄ receptor inhibits the enzyme adenylate cyclase, thereby reducing intracellular cAMP. The D₄ receptor is encoded by the DRD4 gene (e.g. FIG. 8A). As used herein, DRD4 encompasses mammalian DRD4, including the human receptor (FIG. 8B), functionally equivalent variants and isoforms thereof, as well as non-human DRD4, e.g. non-human species such as mouse (FIG. 8C/D), rat, dog, cat, etc. The term “functionally equivalent” refers to variants and isoforms of the DRD4 receptor that essentially retain function as a D4 dopamine receptor, e.g. retain ligand binding activity. The term “functionally equivalent” is used herein to refer to a D₄ receptor protein that exhibits the same or similar function to the native protein (retains at least about 50% of the activity of the human receptor), and includes all isoforms, variants (e.g. Val194Gly), and other naturally-occurring forms. The term “functionally equivalent” also refers to nucleic acid, e.g. mRNA, DNA or cDNA, encoding the D₄ receptor, and is meant to include any nucleic acid sequence that encodes a functional D₄ receptor, including all transcript variants, variants that encode protein isoforms, variants due to degeneracy of the genetic code, and the like. Protein modifications may include, but are not limited to, one or more amino acid substitutions, additions or deletions; modifications to amino acid side chains; and the like. Nucleic acid modifications may include one or more base differences due to degeneracy of the genetic code or sequence differences Which encode D₄ variants such as variants incorporating a 48-base pair variable number tandem repeat (VNTR) in exon 3 (e.g. 2-11 repeats), C-521T in the promoter, 13-base pair deletion of bases 235 to 247 in exon 1, 12 base pair repeat in exon I, or a polymorphic tandem duplication of 120 bp.

Antagonists of the dopamine D₄ receptor include compounds that inhibit or prevent the activity of the D₄ receptor, for example, by inhibiting the interaction, such as binding interaction at a binding or active site, of the receptor with its endogenous ligand or substrate. Examples of dopamine D₄ receptor antagonists include, but are not limited to, A-381393, L-745,870, L-750,667, L-741,742, S 18126, fananserin, clozapine, buspirone, FAUC 213, sonepiprazole, PD 168568 dihydrochloride and PNU 96415E. Preferred antagonists include antagonists which are specific for DRD4 such as L-741,742 and PNU 96415E.

The term “neuronal” or “neural” stem cell refers to self-renewing, multipotent cells that generate the main phenotype of the nervous system. Neural stem cells primarily differentiate into neurons, astrocytes, and oligodendrocytes. Neurons are further classified by the neurotransmitter with which they interact, e.g. glutamate, GABA (gamma-aminobutyric acid), dopamine, serotonin and acetylcholine.

As one of skill in the art will appreciate, dopamine D₄ receptor antagonists may be formulated for use in accordance with the present invention. Thus, the selected antagonist may be formulated by combination with a pharmaceutically acceptable carrier. The expression “pharmaceutically acceptable” means acceptable for use in the pharmaceutical and veterinary arts, i.e. not being unacceptably toxic or otherwise unsuitable. As one of skill in the art will appreciate, the selected carrier will vary with the administrable route used. In this regard, the selected antagonist may be administered by any suitable route. In one embodiment, the selected antagonist is formulated for administration by infusion or injection, either subcutaneously or intravenously, and thus, may accordingly be utilized in combination with a medical-grade carrier, such as an aqueous solution in sterile and pyrogen-free form, optionally buffered or made isotonic. Thus, suitable carriers include distilled water or, more desirably, a sterile carbohydrate-containing solution (e.g. sucrose or dextrose, such as a 5% dextrose solution) or a sterile saline solution comprising sodium chloride and optionally buffered. Suitable sterile saline solutions may include varying concentrations of sodium chloride, for example, normal saline (0.9%), half-normal saline (0.45%), quarter-normal saline (0.22%), and solutions comprising greater amounts of sodium chloride (e.g. 3%-7%, or greater). Saline solutions may optionally include additional components, e.g. carbohydrates such as dextrose and the like. Examples of saline solutions including additional components, include Ringer's solution, e.g. lactated or acetated Ringer's solution, phosphate buffered saline (PBS), TRIS (hydroxymethyl) aminomethane hydroxymethyl) aminomethane)-buffered saline (TBS), Hank's balanced salt solution (HBSS), Earle's balanced solution (EBSS), standard saline citrate (SSC), HEPES-buffered saline (HBS) and Gey's balanced salt solution (GBSS).

In other embodiments, the selected antagonist may be formulated for administration by routes including, but not limited to, oral, intraperitoneal, intranasal, enteral, topical, sublingual, intramuscular, intra-arterial, intramedullary, intrathecal, inhalation, ocular, transdermal, vaginal or rectal routes, and will be combined with appropriate carriers in each case. For example, compositions for oral administration via tablet, capsule or suspension may be prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Compositions for topical application may be prepared including appropriate carriers. Creams, lotions and ointments may be prepared for topical application using an appropriate base such as a triglyceride base. Such creams, lotions and ointments may also contain a surface active agent. Aerosol formulations may also be prepared in which suitable propellant adjuvants are used. Other adjuvants may also be added to the composition regardless of how it is to be administered, for example, anti-microbial agents may be added to the composition to prevent microbial growth over prolonged storage periods.

In the present method of utilizing a dopamine D₄ receptor antagonist for neuronal stem cell differentiation, the selected antagonist is administered to mammalian neuronal stem cells, in vitro, ex vivo or in vivo, in a therapeutically effective amount to promote differentiation of the stem cells. The term “mammal” and “mammalian” is used herein to encompass human and non-human mammals, including domesticated animals such as dogs, cats, horses and the like; an undomesticated animals. The term “therapeutically effective amount” is an amount of DRD4 antagonist required to promote neuronal stem cell differentiation, while not exceeding an amount which may cause significant adverse effects to the stem cells, i.e. undesirable cell changes or effect on cell growth, or cell death. DRD4 antagonist dosages that are therapeutically effective may vary with the neuronal stem cells to be treated, and the type of differentiated cell to be achieved. In one embodiment, dosages within the range of about 0.1-100 mg/m² is appropriate for use to promote differentiation of neuronal stein cells into glutamergic or GABAergic cells, for example, 0.5-50 mg/m², e.g. 1-10 mg/m².

The present method of promoting differentiation of neuronal stein cells may be therapeutically applied to treat conditions in a mammal involving a defect in a particular type of neuron. The terms “treat”, “treating” or “treatment” are used herein to refer to methods that favorably alter the target pathological condition, including those that moderate, reverse, reduce the severity of, or protect against, the progression of the target disorder. For example, use of dopamine D₄ receptor antagonists to promote differentiation of glutamatergic neurons is useful to treat pathologic conditions involving these neurons, e.g. neurodegenerative disorders, including but not limited to, Parkinson's and Alzheimer's, traumatic brain injury (including mechanical head injury and surgery), ischemic or hemorrhagic stroke, seizures and metabolic or congenital disorders resulting from deficiencies in glutamatergic neurons.

In another embodiment, dopamine D4 receptor antagonists to promote differentiation of GABAergic neurons is useful to treat pathologic conditions in a mammal involving these neurons, e.g. anxiety, obsessive compulsive, and mood disorders including major depressive disorder, bipolar disorder and seasonal affective disorder, neurodegenerative disorders including Parkinson's disease (PD) and PD-related disorders, Alzheimer's disease and other dementias, Huntington's disease, motor neurone disease, spinocerebellar ataxia, autism and spinal muscular atrophy, epilepsy, seizures, traumatic brain injury, and ischemic stroke.

Embodiments of the present invention are illustrated in the following specific example which is not to be construed as limiting.

### EXAMPLE 1

### Materials and Methods

Differentiation medium. Step-I medium—Neurocult NS-A basal medium supplemented with 1×B27 and 1×N2 with basic fibroblast growth factor (FGF2-5 ng/ml) and no epidermal growth factor (EGF). Step-II medium—Neurocult NS-A basal media mixed with Neurobasal media (1:1) with N2 (¼ amount) and B27 (no EGF and FGF).

High content screen. Human fetal NSC (hf5205) cells (cells derived from fetal brain tissues approximately 10 weeks old) were seeded at 1000 cells/well in a 384 well plate coated with Poly-L-ornithine (PLO) and laminin. Neuroactive compounds from a compound library were added to each well at a concentration of 5 μM in the step-I medium (without EGF) and incubated at 37° C. and 5% CO₂. At day 4 of the incubation, the medium was changed to step-II medium (without FGF) and the cells were incubated with the same test compound for another 4 days. At day 8, cells were fixed with 4% paraformaldehyde and immunostained to identify a neuronal marker (Beta III tubulin), astrocytic marker (Glial Fibrillary Acidic Protein—GFAP) and DAPI. Images were taken using Evotek Opera system. 30 images were taken from different areas of each well and the data was analyzed using Acapella Script program using average signal intensity with set cut-offs and DAPI as a reference for a cell. Bone morphogenetic protein (BMP4) and BIO (Glycogen synthase kinase-3 inhibitor) were used as positive controls for differentiation.

HfNS (hf5205) cells was seeded in 384-well black μ clear bottom coated with laminin and poly-L-ornithine (PLO) at a density of 1×10³. Compound library was added at approximately 5 μM using Biomek FX^{P} automation workstation with pin tool. Cells were incubated in Step-I medium (N2, B27, 5 ng/ml FGF) without EGF for the first four days and replaced with step-II medium (1:1 Neurobasal:Neurocult, B27, ¼ N2) without FGF for the next four days. At day 8, cells were fixed with 4% paraformaldehyde (PFA) and blocked with 5% normal goat serum (NGS) and incubated with primary antibody against anti-Beta III tubulin (1:100 MAB1637), anti-GFAP (1:1000, DAKO) and DAPI overnight at 4° C. 25 images were taken from different areas of each well by Evotek Opera system and data were analyzed using Acapella software program using average signal intensity with set cut offs using DAPI stain as reference for cells. BMP4 and BIO (GSK3b inhibitor) were used as a positive control for differentiation.

Immunocytochemistry. hfNS cells differentiated on coverslips were fixed with 4% paraformaldehyde and permeabilized with 0.3% Triton X100, block with 5% goat serum, and incubated with primary antibody overnight at 4° C. Antibodies included: Beta-III tubulin (1:100 MAB1637), GFAP (1:1000, DAKO), Vglut1 (1:1000, Synaptic system), GABA (1:1000, Sigma). Appropriate fluorescent-conjugated secondary antibodies were used at 1:500 for 1 h at room temperature. Coverslips were mounted with fluorescent mounting medium (DAKO) containing DAPI (1:1000) and cells were imaged using Leica microscope.

Hit validation for differentation. All retests for differentiation were done by a two step growth withdrawal method (as described in Sun et al. Mol. Cell. Neurosci. 38(2008) 245-258) with and without the test compound for 8 days. Test compounds included L-741,742 (3 μM), PNU96415E (15 μM), Ifenprodil tartrate (1.5 μM), McN A-343 (4 μM), and (−) —N-Phenylcarbamoyleseroline (6 μM). For mature neuronal marker analysis, human fetal NSC were differentiated for 3 weeks using L-741,742 (2-4 μM varying at different stages) in the two step growth withdrawal method.

Single Cell qPCR (Fluidigm Analysis). Differentiated hfNS (hf5205) at week 3 was accutased and cells were resuspended in NS medium containing 1 μg per ml of propidium iodide and filtered through a 40 μm nylon cell strainer followed by live single cell sorting into a 96 well qPCR plate on a BD cell sorter. Cells were sorted into 10 μl of preamplification mix containing 40 nM of all primers for the 48 genes, and the following components of CellsDirect One-Step qRT-PCR kit (Invitrogen) as directed in protocol: 2× reaction mix, SuperScript III RT/platinum Taq mix. After sorting, samples were reverse transcribed and preamplified for 18 cycles. Preamplified samples were diluted (2×) with TE buffer and stored at −20° C. Sample and assay primer preparation for Fluidigm Dynamic arrays was done according to the manufacturer's recommendation. Samples were mixed with 2× assay loading reagent (Fluidigm Corp), 20× EvaGreen and TaqMan gene expression master mix. The Fluidigm Dynamic arrays were primed and loaded on the IFC controller and qPCR experiments were run on a Biomark system for genetic analysis.

Fluidigm Analysis. Hf5205 cells were differentiated in the two-step growth withdrawal method, with and without L-741,742 (3.5 μM) for three weeks. The differentiated cells were harvested and filtered through a 40 uM nylon filter and live sort single cell (based on PI staining) into a 96 well qRT-PCR plate containing pre-amplification mix of 48 primers, reverse transcribed and amplified for 48 genes (as described in Pasca et al. Nature Medicine (2011) November 27; 17(12) 1657-62) in one step using a kit from Invitrogen. Samples and primer pairs were prepared for Fluidigm as per protocol. Cells were identified based on presence of RPS18 and GAPDH, Data was analyzed based on population of cells expressing particular genes using different CT value cut off.

Gene Expression Profiling. Hf5205 cells were differentiated with 3 μM of L-741,742 in a two-step growth factor withdrawal protocol. RNA was extracted using an RNAeasy kit (Qiagen) from control and treated samples at each time point 0 h (NSC), 48 h after treatment (-EGF), 9 days of treatment (-FGF) and 3 weeks of treatment. RNA extracted from the samples was hybridized on Affymetrix Human Gene 1.0 ST arrays using standard protocol (TCAG, Toronto, Ontario, Canada). RMA background correction, quantile normalization and log2 transformation were applied to the CEL files using the Bioconductor ally package (R 3.0.1, affy package version 1.38.1). Batch correction was applied using ComBat function from sva (3.6.0) and gene annotations were retrieved using hugene10sttranscriptcluster.db (8.0.1). Genes were ranked based on the average log fold change (log FC) of the L-741,742 differentiated hfNS and control differentiated hfNS at 48 h, 9 days and 3 weeks. The data were analyzed using GSEA (Subramanian et al., Proc. Natl. Acad. Sci. 2005. 102(43), 15545-15550) with parameters set to 2000 gene-set permutations and gene-sets size between 8 and 500. The gene-sets included in the GSEA analyses were obtained from KEGG, MsigDB-c2, NCI, Biocarta, IOB, Netpath, HumanCyc, Reactome and the Gene Ontology (GO) databases, updated Oct. 14, 2013 (http://baderlab.org/GeneSets). An enrichment map (version 1.2 of Enrichment Map software (Mexico et al., 2010. PLoS One 5(11), e13984) was generated for each comparison using enriched gene-sets with a False Discovery Rate <0.02% and the overlap coefficient set to 0.5.

### Results

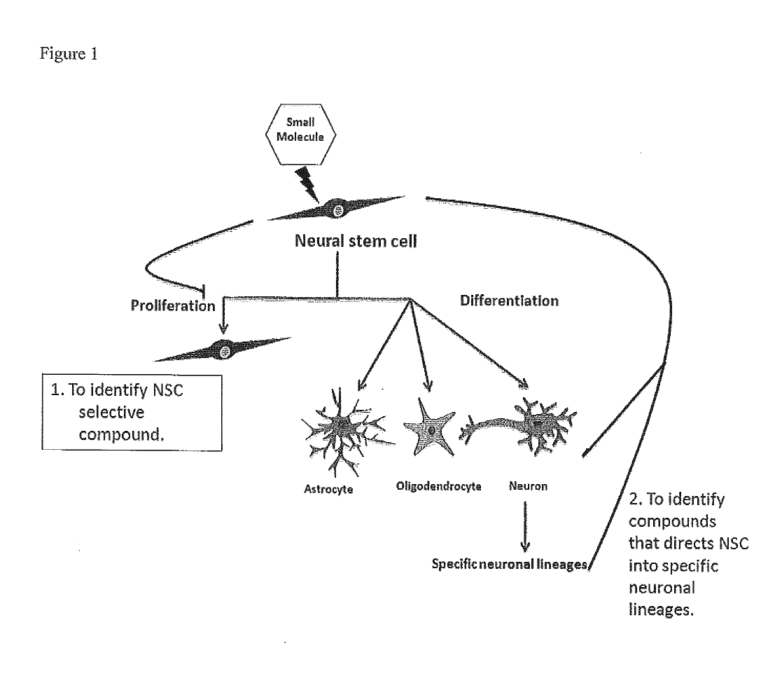

Identification of compounds that can direct NSC fate. A 680-neuroactive compound library was interrogated in human fetal NSC for differentiation potential. The differentiation screen was conducted in a 8 day time period (4+4) to identify compounds that accelerate the differentiation mechanism compared to a default differentiation method of human fetal NSC which takes 3 weeks in a two-step growth withdrawal medium. Twenty two (22) compounds were identified that showed an increase in beta III tubulin staining indicating neuronal differentiation and 17 compounds were identified that showed GFAP staining indicating astrocytic differentiation (FIG. 1). The top hits were validated and five compounds including McN A-343, (−) —N-phenylcarbamoyleseroline, ifenprodil tartrate, L741742 and PNU96415E were confirmed to promote neuronal differentiation ranging from 12-40% neuronal differentiation when compared to the control (2-9%), L741742 and PNU96415E were initially identified as NS selective compounds.

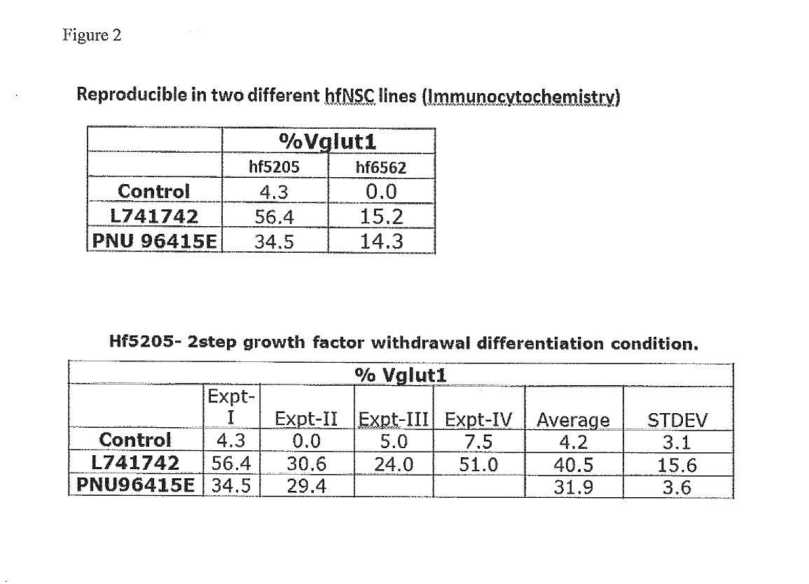

DRD4 antagonists differentiate human fetal NSC into specific glutamatergic lineages. To determine if the hit compounds have the potential to specify specific neuronal lineages, the human fetal NSC (hf5205) was differentiated with the hit compounds for 3 weeks using the 2-step growth withdrawal methods. Immunocytochemistry was performed for a series of antibodies marking different classes of neurons based on their neurotransmitters including glutamatergic, dopaminergic, cholinergic and serotonergic. Interestingly, three of the compounds including L-741,742, PNU96415E and Ifenprodil tartrate showed Vglut1-positive staining indicating differentiation into glutamatergic neurons. Vglut1 is a vesicular glutamate transporter required for transport of glutamate into synaptic vesicles of glutamatergic neurons. Two of the hit compounds L-741,742 and PNU96415E are dopamine D4 receptor antagonists showing an average of 30-40% of Vglut1 positive staining compared to control (0-4.2%). Differentiation potential of L-741,742 was further validated in two other human fetal NSC lines (hf6562 and hf6539) and similar potential to promote Vglut1 positive glutamatergic neuron differentiation was found (FIG. 2).

The differentiated cells were further tested for gene expression of different neuronal lineage markers by RT-qPCR. A series of primers were designed for markers representing both neurotransmitters and domain specific regions as shown in Table 1.

TABLE 1
Primers used in RT-qPCR assays.

| Primer Name | Sequence | SEQ ID NO: |
|---|---|---|
| CTIP2-F | TCCGAGCCGGTGGAGATCGG | 3 |
| CTIP2-R | GCACGGCCCTGCAATGTTCTC | 4 |
| FOXG1-F | CGGCTCCCTCTACTGGCCCA | 5 |
| FOXG1-R | ATGGGGTGGCTGGGGTAGGC | 6 |
| VGLUT1-F | GGCCAGATCGCGGACTTCCT | 7 |
| VGLUT1-R | CAACAGCAGCGTGGCTTCCA | 8 |
| NEUROG2-F | ACCACAAGCAGCTTCGCGTTA | 9 |
| NEUROG2-R | CGGGTCTCGTGTGTTGTGGTG | 10 |

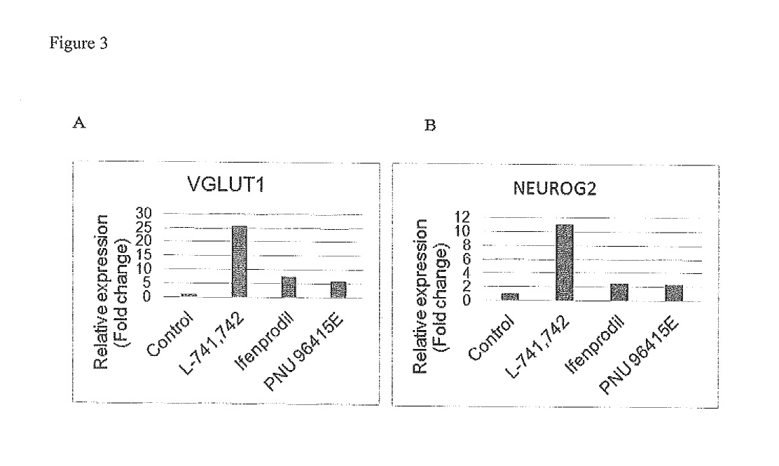

The L-741,742-differentiated cells showed 15-35 fold increase in VGLUT1 expression, 11-fold increase in NEUROG2 expression when compared to the control (FIG. 3). This further confirms that L-741,742 promotes differentiation into glutamatergic neurons (Vglut1+). Neurog2 or neurogenin2 is a proneural marker that promotes glutamatergic excitatory neurons in the dorsal telencephalic region during cortical development.

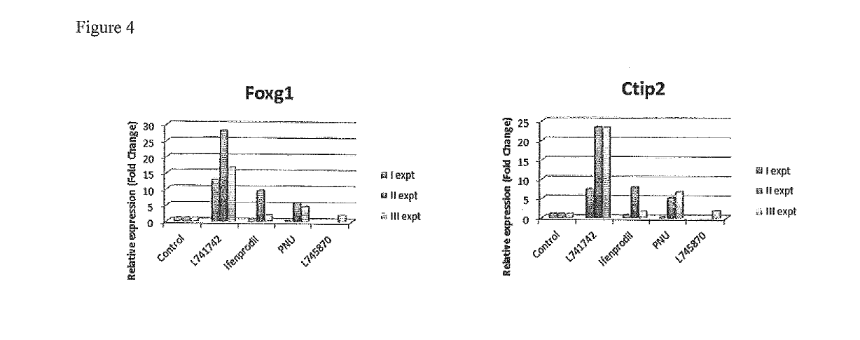

L-741,742-differentiated neurons show cortical layer V marker. L-741,742-differentiated cells show increased expression of VGLUT1 and NEUROG2. NEUROG2 expression is required for promoting excitatory neurons in the dorsal cortical region during development. To test if L-741,742 promotes or specifies specific cortical layer neurons, a series of primers were designed to mark all layers of cortex; Reelin (layer 1), Tbr1 (layer V1), Ctip2 (Layer V), Satb2 (Layer II-III) and Foxg1 (telenchepalic marker) (Table 1). The expression of these markers in L-741,742-differentiated cells was determined by RT-qPCR. An approximately 23-fold increase in CTIP2 expression and 27-fold increase in FOXG1 expression was observed in L-741,742-differentiated cells (FIG. 4). Reelin and Tbr1 expression were not detectable at the concentration tested. Thus, L-741,742-differentiated neurons express cortical layer V marker CTIP2 and telencephalic marker FOXG1, with VGLUT1 expression, indicating differentiation into cortical layer neurons.

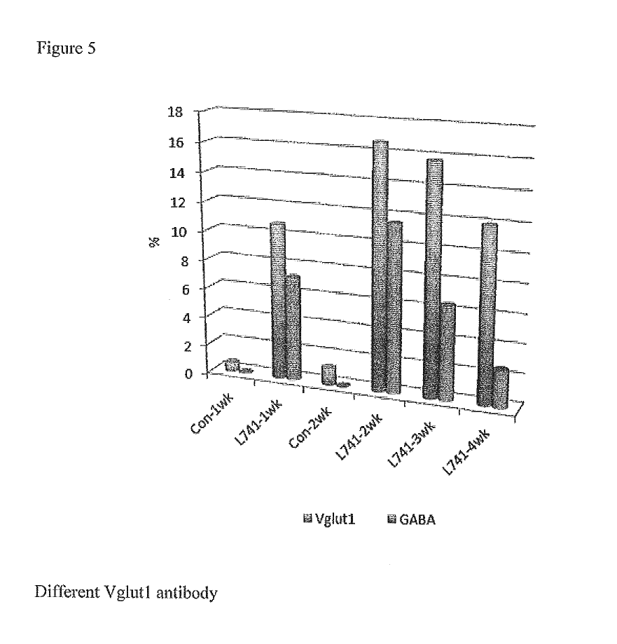

L-741,742-differentiated cells also showed GABA positive neurons. L-741,742-differentiated cells were also tested for GABA staining from week 1-3 and GABA positive cells were present in early in 1 week differentiated cells, showing up to 11% GABA positive neurons in 2 week differentiated cells (FIG. 5). Thus, L-741,742-differentiated cells showed the presence of both glutamatergic neurons and GABAergic neurons.

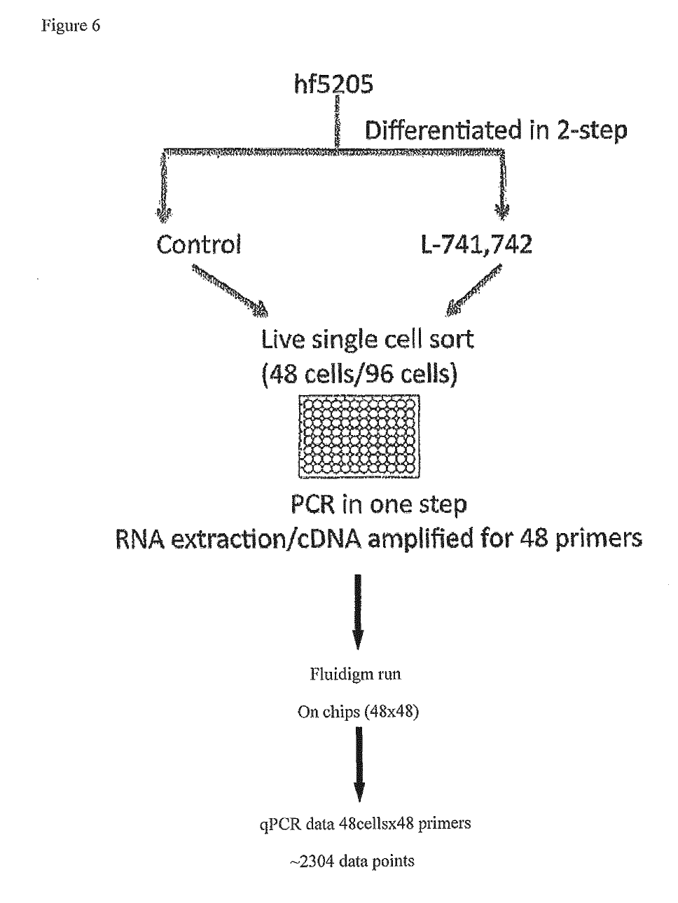

Characterization of L-741,742-differentiated cells by Fluidigm analysis. To further characterize the different population of neurons in L-741,742 differentiated culture, a single cell Fluidigm array was employed that can perform multiple qRT-PCR (48 genes or 96 genes) at a single cell level as shown in FIG. 6. Forty eight (48) gene primers marking various cell populations were used including, early progenitor marking dorsal, ventral or midbrain, differentiated neuronal markers based on neurotransmitters GABA or glutamatergic and markers specifying different cortical layers. Primer sequences were obtained from Pasca et al. Nature Medicine (2011) November 27; 17(12) 1657-62).

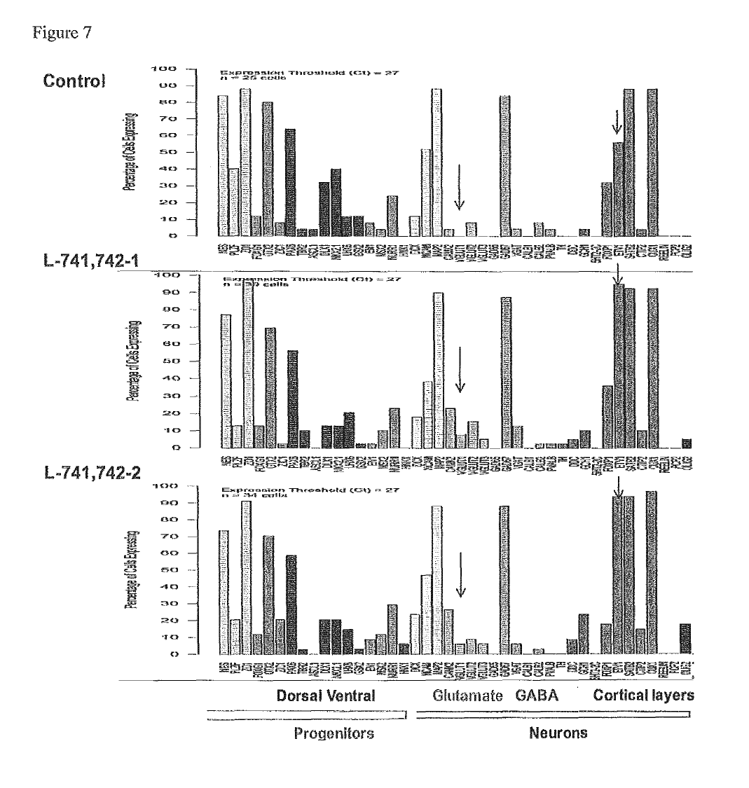

An increase in the expression of MAP2 (marking neurons), EVT1 (marking lower cortical layer V), and CAMKII, VGLUT1, VGLUT2 and VGLUT3 (marking glutamatergic neurons) in the L-741,742-differentiated cells (FIG. 7). Thus, L-741,742-differentiated cells showed an increase in an ETV1-expressing cell population marking lower layer marker V, and CAMKII-, VGLUT1-, VGLUT2- and VGLUT3-expressing cells marking glutamatergic neurons. The increase in ETV1 and CAMKII were further validated by qRT-PCR of total RNA from the same culture.

Relevant portions of references referred to herein are incorporated by reference.

1. A method of promoting mammalian neural stern cell differentiation comprising exposing neural stem cells to a dopamine D₄ receptor antagonist.

2. The method of claim 1, wherein the dopamine D₄ receptor antagonist is selected from the group consisting of A-381393, L-745,870, L-750,667, L-741,742, S 18126, fananserin, clozapine, buspirone, FAUC 213, sonepiprazole, PD 168568 dihydrochloride and PNU 96415E.

3. The method of claim 1, to promote differentiation into glutamatergic cells.

4. The method of claim 1, to promote differentiation into GABAergic cells.

5. The method of claim 1, wherein the neural stems cells are treated with dopamine D₄ receptor antagonist in an amount in the range of about 0.1-100 mg/m².

6. The method of claim 5, wherein the neural stems cells are treated with dopamine D₄ receptor antagonist in an amount in the range of about 0.5-50 mg/m².

7. The method of claim 6, wherein the neural sterns cells are treated with dopamine D₄ receptor antagonist in an amount in the range of about 1-10 mg/m².

8. The method of claim 2, wherein the dopamine D₄ receptor antagonist is L-741,742 or PNU96415E.

9. A method of treating a pathological condition in a mammal comprising administering to the mammal a dopamine D₄ receptor antagonist, wherein the pathological condition is a condition resulting from a deficiency in glutamatergic neurons or GABAergic neurons.

10. The method of claim 9, wherein the dopamine D₄ receptor antagonist is selected from the group consisting of A-381393, L-745,870, L-750,667, L-741,742, S 18126, fananserin, clozapine, buspirone, FAUC 213, sonepiprazole, PD 168568 dihydrochloride and PNU 96415E.

11. The method of claim 9, wherein the pathological condition is a condition resulting from a deficiency in glutamatergic neurons and is selected from the group consisting of Parkinson's disease, Alzheimer's, traumatic brain injury, ischemic or hemorrhagic stroke, seizures and metabolic or congenital disorders resulting from deficiencies in glutamatergic neurons.

12. The method of claim 9, wherein the pathological condition is a condition resulting from a deficiency in GABAergic neurons selected from the group consisting of anxiety and related mood disorders, neurodegenerative disorders, autism, spinal muscular atrophy, epilepsy, seizures, traumatic brain injury and ischemic stroke.

13. The method of claim 9, wherein the mammal is treated with dopamine D₄ receptor antagonist in an amount in the range of about 0.1-100 mg/m².

14. The method of claim 10, wherein the dopamine D₄ receptor antagonist is L-741,742 or PNU96415E.
